(19) United States
(12) Patent Application Publication
(10) Pub. No.: US 2023/0355420 A1
(43) Pub. Date: Nov. 9, 2023

(54) NASAL MOLDING DEVICE

(71) Applicant: NEW YORK UNIVERSITY, New York, NY (US)

(72) Inventors: Pradip R. Shetye, New York, NY (US); Buddhathida Wangsrimongkol, Khon Kaen (TH)

(21) Appl. No.: 18/297,160

(22) Filed: Apr. 7, 2023

Related U.S. Application Data
(63) Continuation-in-part of application No. PCT/US21/54133, filed on Oct. 8, 2021.
(60) Provisional application No. 63/089,447, filed on Oct. 8, 2020.

(51) Int. Cl. A61F 5/08

(52) U.S. Cl. CPC A61F 5/08

(57) ABSTRACT

The present invention provides individual nasal molding devices and methods of making and using the same. This novel technique of pre-surgical nasal molding therapy is ideal for all patients with unilateral and bilateral clefts, including medically compromised patients or patients living far from the cleft centers and periodic office visits are not practical. It is also ideal for patients with incomplete clefts with significant nasal deformity without the involvement of the alveolus or the palate. Post-surgery, the devices can also be used as a nostril retainer to prevent scar contracture and relapse of the primary correction of the nose. The nasal molding devices can be customized for each infant or be provided in standard sizes for ease of utilization and mass production.

### CROSS-REFERENCE TO RELATED APPLICATIONS

This application is a continuation-in-part of International Patent Application No. PCT/US21/54133, filed on Oct. 8, 2021, which claims priority to U.S. Provisional Patent Application No. 63/089,447, filed Oct. 8, 2020, the contents of which are each incorporated by reference herein in their entirety.

### BACKGROUND OF THE INVENTION

Presurgical NasoAlveolar Molding (NAM) is one of the widely used presurgical infant orthopedics (PSIO) therapy in patients with clefts (Grayson B H et al., The cleft palate-Craniofacial journal. 1999 November; 36(6):486-98). The benefits of NAM includes alignment of the displaced alveolar segments and improvement in the columella length, nasal symmetry and projection prior to primary repair surgery (Garfinkle J S et al., Plastic and reconstructive surgery. 2011 Apr. 1; 127(4):1659-67). NAM therapy can be offered to families willing to undergo the treatment as well as those who can periodically travel to the office for routine adjustments. Infants with underlying medical conditions such as congenital heart disease, failure to thrive, aspiration and feeding disorders are not indicated for NAM therapy (Poonsak Pisek D D et al., J Med Assoc Thai. 2014; 97(10):S17-24).

Lip taping was first used as an extra-oral PSIO to reduce the size of the cleft by stretching lip muscles and skin surfaces as well as preventing further widening from the tongue force (Brown G V, Journal of the American Medical Association. 1905 Mar. 18; 44(11):848-59). In certain situations lip tapes are used as a non-surgical alternative to lip adhesion procedure (Pool R et al., Annals of plastic surgery. 1994 Mar. 1; 32(3):234-49). Lip taping is easy to use and it can be somewhat effective in bringing the cleft maxillary alveolar segments together (Dawjee S M et al., South African Dental Journal. 2014 Mar. 1; 69(2):62-9). However, lip taping lacks the nasal molding element that helps to reposition and reshape the displaced nasal cartilages. Berggren et al. (Plastic and reconstructive surgery. 2005 May 1; 115(6):1785-7) proposed a nasal alar elevator consisting of a stainless-steel wire and silicone tube secured with surgical tape to the forehead to improve nasal asymmetry. Later, Monasterio et al. (Monasterio L et al., The Cleft Palate-Craniofacial Journal. 2013 September; 50(5):548-54) refined and developed a paper tape with elastic band called Dynacleft® (Canica Design Inc., Almonte, Ontario, Canada) and a nasal elevator component called Dynacleft® Nasal Evelator System (DNES). The DNES is attached to the infant's forehead with a Dynacleft® with a upward and backward force vectors (Enlow D H et al., Essentials of facial growth. WB Saunders Company; 1996). This appears to be counterintuitive force vector as normal nasal growth is in the forward and downward direction. Other authors have modified nasal springs with lip taping to mold nasal cartilages without an intra-oral acrylic plate (Wang Q et a l., Plastic and Reconstructive Surgery Global Open. 2013 July; 1(4); Peanchitlertkajorn S, The Cleft Palate-Craniofacial Journal. 2019 February; 56(2):280-4), but lack effectiveness in molding of the nose due to lack of a reciprocal force.

Computer-assisted design and computer-aided manufacturing (CAD/CAM) technology has become an essential tool in the medical and dental fields (Gerstle T L et al., Plastic and reconstructive surgery. 2014 Feb. 1; 133(2):446-51; Zinser M J et al., Journal of oral and maxillofacial surgery. 2013 Dec. 1; 71(12):2151-e1). It makes treatment customization possible. Lou et al. (The Cleft Palate-Craniofacial Journal. 2019 April; 56(4):521-4) used 3D printing technology to personalized nasal stents for adult patients with CLP. Furthermore, several authors have utilized CAD/CAM technology to streamline the PSIO therapy by developing 3D-printed NAM appliances as well as developed treatment algorithm (Grill F D et al., Scientific Reports. 2018 Aug. 7; 8(1):1-8; Grill F D et al., Scientific reports. 2018 Aug. 14; 8(1):1-9; Bauer F X et al., Biomedical Engineering/Biomedizinische Technik. 2017 Aug. 28; 62(4):407-14; Schiebl J et al., IEEE Transactions on Biomedical Engineering. 2019 Aug. 12; 67(5):1263-71). However, an optimal and predictable PSIO treatment alternative for medically compromised patients with CL/P still does not exist.

Thus, there is a need in the art for improved devices for molding devices for cleft lip and palate repair. The present invention meets this need.

### SUMMARY OF THE INVENTION

The present invention relates to a nasal molding device, comprising: a pair of nasal molding stents comprising a first and a second molding stent, each molding stent having a substantially tubular shape comprising a superior end extending for a length to an inferior end; a columella bridge laterally attached to the inferior end of each molding stent, the columella bridge having a width; and a lip band attached to the columella bridge and the inferior end of each molding stent, the lip band having a substantially planar shape comprising a length extending between opposing lateral ends, a height extending from a superior to an inferior direction, and a thickness; wherein each molding stent comprises a molding stent axis aligned in parallel with the length of each molding stent, and the lip band comprises a lip band axis aligned in parallel with the width of the lip band.

In one embodiment, each molding stent comprises a superior width and an inferior width. In one embodiment, the superior width and the inferior width are each between about 1 and 15 mm. In one embodiment, the inferior width of the first molding stent is equal to the inferior width of the second stent. In one embodiment, the inferior width of the first molding stent is not equal to the inferior width of the second stent. In one embodiment, the length of each molding stent is between about 5 and 15 mm. In one embodiment, the superior end of each molding stent has a Shore hardness that is less than the inferior end of each molding stent. In one embodiment, the Shore hardness of the superior end is between about 0 Shore OO and 100 Shore OO. In one embodiment, the Shore hardness of the inferior end is between about 0 Shore OO and 100 Shore OO.

In one embodiment, the molding stent axis and the lip band axis form an angulation between about 90° and 180°, such that each molding stent is angled in a posterior direction. In one embodiment, the angulation is between about 110° and 160°. In one embodiment, the molding stent axis of the first molding stent is aligned in parallel with the molding stent axis of the second molding stent. In one embodiment, the molding stent axis of the first molding stent is not aligned in parallel with the molding stent axis of the second molding stent.

In one embodiment, the width of the columella bridge is between about 1 and 15 mm.

In one embodiment, the length of the lip band is between about 20 and 100 mm. In one embodiment, the height of the lip band is between about 1 and 15 mm. In one embodiment, the thickness of the lip band is between about 0.5 and 5 mm. In one embodiment, each lateral end comprises a loop. In one embodiment, the height of the lip band curves in a superior direction at a position directly inferior to an attachment between the lip band and the columella bridge. In one embodiment, the length of the lip band comprises a curvature, such that each lateral end curves in a posterior direction. In some aspects, the present invention relates to a method having the steps of providing a first nasal molding device having a pair of nasal molding stents having a first and a second molding stent, each molding stent having a substantially tubular shape having a superior end extending for a length to an inferior end, a columella bridge laterally attached to the inferior end of each molding stent, the columella bridge having a width, and a lip band attached to the columella bridge and the inferior end of each molding stent, the lip band having a substantially planar shape having a length extending between opposing lateral ends, a height extending from a superior to an inferior direction, and a thickness, wherein each molding stent has a molding stent axis aligned in parallel with the length of each molding stent, and the lip band has a lip band axis aligned in parallel with the width of the lip band, and affixing the first device to a subject.

In some embodiments, the method further has the steps of removing the first device from the subject, providing a second nasal molding device having a pair of nasal molding stents having a first and a second molding stent, each molding stent having a substantially tubular shape having a superior end extending for a length to an inferior end, a columella bridge laterally attached to the inferior end of each molding stent, the columella bridge having a width, and a lip band attached to the columella bridge and the inferior end of each molding stent, the lip band having a substantially planar shape having a length extending between opposing lateral ends, a height extending from a superior to an inferior direction, and a thickness, wherein each molding stent has a molding stent axis aligned in parallel with the length of each molding stent, and the lip band has a lip band axis aligned in parallel with the width of the lip band, affixing the second device to the subject, wherein the columella bridge width of the second device is less than the columella bridge width of the first device, the distance between the superior ends of the molding stents of the second device is less than the distance between the superior ends of the molding stents of the first device, or the wall thicknesses of the pair of nasal molding stents of the second device is greater than the wall thicknesses of the pair of nasal molding stents of the first device.

In some embodiments, the method further has the steps of providing one or more retainers, each retainer having a planar shape having a top surface having one or more connectors and a bottom surface configured to adhere to the subject's face or head, adhering the one or more retainers to the skin of the subject, and attaching one or more elastic bands from the one or more connectors of the retainers to the nasal molding device.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1 depicts a front view of an exemplary symmetrical individual nasal molding device.

FIG. 2 depicts side views of an exemplary individual nasal molding device.

FIG. 3A through FIG. 3Q depicts additional views of exemplary individual nasal molding devices. FIG. 3A depicts a bottom view of an exemplary symmetrical individual nasal molding device (top) and a bottom perspective view of an exemplary asymmetrical individual nasal molding device (bottom). FIG. 3B depicts an exemplary individual nasal molding device comprising button connectors (top, middle) and an exemplary individual nasal molding device comprising hook connectors (bottom).

FIG. 3C depicts various views of an exemplary individual nasal molding device according to aspects of the present invention. FIG. 3D depicts a front view an exemplary individual nasal molding device (left) and an enlarged cross-sectional side view of an exemplary individual nasal molding device (right). FIG. 3E depicts a side view of an exemplary individual nasal molding device (left) and an enlarged cross-sectional front view of an exemplary individual nasal molding device (right). FIG. 3C depicts various views of an exemplary individual nasal molding device according to aspects of the present invention. FIG. 3D depicts a front view an exemplary individual nasal molding device (left) and an enlarged cross-sectional side view of an exemplary individual nasal molding device (right). FIG. 3E depicts a side view of an exemplary individual nasal molding device (left) and an enlarged cross-sectional front view of an exemplary individual nasal molding device (right). FIG. 3F depicts various views of an exemplary individual nasal molding device according to aspects of the present invention. FIG. 3G depicts a front view an exemplary individual nasal molding device (left) and an enlarged cross-sectional side view of an exemplary individual nasal molding device (right). FIG. 3H depicts a side view of an exemplary individual nasal molding device (left) and an enlarged cross-sectional front view of an exemplary individual nasal molding device (right). FIG. 3I depicts various views of an exemplary individual nasal molding device according to aspects of the present invention. FIG. 3J depicts a front view an exemplary individual nasal molding device (left) and an enlarged cross-sectional side view of an exemplary individual nasal molding device (right). FIG. 3K depicts a side view of an exemplary individual nasal molding device (left) and an enlarged cross-sectional front view of an exemplary individual nasal molding device (right). FIG. 3L depicts a top view (top) and a side view (bottom) of an exemplary elastic retainer comprising a plurality of retainer hooks according to aspects of the present invention. FIG. 3M depicts a top perspective view (top) and a bottom perspective view (bottom) of an exemplary elastic retainer comprising a plurality of retainer hooks. FIG. 3N depicts various views of an exemplary elastic retainer according to aspects of the present invention. FIG. 3O depicts a side view an exemplary elastic retainer (bottom) and an enlarged side view of an exemplary elastic retainer (top). FIG. 3P depicts a front view of an exemplary elastic retainer (bottom) and an enlarged cross-sectional side view of an exemplary elastic retainer (top). FIG. 3Q depicts a front view an exemplary individual nasal molding device comprising elastic retainers with elastic bands according to aspects of the present invention.

FIG. 4 is a table listing the dimensions of exemplary symmetrical individual nasal molding devices.

FIG. 5 depicts various views of a 3D model of an individual nasal molding device.

FIG. 6 is a flowchart depicting a semi-digital workflow for pre-surgical individual nasal molding therapy.

FIG. 7A through FIG. 7F depict images of a patient with unilateral cleft lip and palate (UCLP) treated with individual nasal molding therapy. (FIG. 7A) Initial presentation showing the nasal and alveolar deformity. (FIG. 7B) Four weeks into lip taping therapy and before starting of iNM treatment. (FIG. 7C) Application of horizontal lip tape for alveolar molding. (FIG. 7D) Photograph showing insertion of iNM device with lip taping. Note the vector of the tapes to maintain the iNM device in the nose. (FIG. 7E) Patient at 10 days after iNM therapy. (FIG. 7F) Starting with asymmetric iNM device for overcorrection of the affected left alar cartilage.

FIG. 8A depicts images of a patient with bilateral cleft lip and palate before treatment (top left), during treatment (top right), and after treatment (bottom); the patient shows positive signs of response 10 days into treatment. FIG. 8B depicts images of a patient with unilateral cleft lip and palate before treatment (top left), during treatment (top middle), and after treatment (top right); and a patient with bilateral cleft lip and palate before treatment (bottom left), during treatment (bottom middle), and after treatment (bottom right).

### DETAILED DESCRIPTION

The present invention provides individual nasal molding devices and methods of making and using the same. This novel technique of pre-surgical nasal molding therapy is ideal for all patients with unilateral and bilateral clefts, including medically compromised patients or patients living far from the cleft centers and periodic office visits are not practical. It is also ideal for patients with incomplete clefts with significant nasal deformity without the involvement of the alveolus or the palate. Post-surgery, the devices can also be used as a nostril retainer to prevent scar contracture and relapse of the primary correction of the nose. The nasal molding devices can be customized for each infant or be provided in standard sizes for ease of utilization and mass production.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The terms “proximal,” “distal,” “anterior,” “posterior,” “medial,” “lateral,” “superior,” and “inferior” are defined by their standard usage indicating a directional term of reference. For example, “proximal” refers to an upper location from a point of reference, while “distal” refers to a lower location from a point of reference. In another example, “anterior” refers to the front of a body or structure, while “posterior” refers to the rear of a body or structure. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Individual Nasal Molding Device

Cleft lip and/or palate (CL/P) is one of the most common congenital anomalies and affects 1/700 live births in the United States every year. The incidence of cleft lip with or without palate is about 4500 new cases per year in the US alone. The present invention relates to orthopedics appliances used to minimize the deformities associated with CL/P before primary surgical repair. Such deformities were wide cleft gap and nasal asymmetry, a so-called “cleft lip nose” deformity. By creating a near “normal” structures, the more predictable and esthetic outcomes can be achieved.

Pre-surgical infant orthopedic (PSIO) can be categorized into extra- and intra-oral appliance with the ultimate goal of reducing post-surgical wound dehiscence from the wide cleft repair. The earlier types of PSIO were focused primarily on approximating cleft maxillary segments prior to primary lip and nasal repair. In the past two decades, the NasoAlveolar Molding (NAM) device was introduced at NYU in 1990 by Barry H Grayson, DDS and Court Cutting, MD, and now has become the gold standard of PSIO therapy. Unlike other previous PSIOs, the NAM device has a nasal extension(s) that is used to mold the distorted nostril(s). Taking advantage of the plasticity of nasal cartilages in newborns, the NAM device allows simultaneous reduction of cleft gap and improvement of nasal symmetry.

Despite several advantages over other PSIO devices, NAM therapy has its limitations. NAM therapy has a steep learning curve that requires experienced orthodontists in the cleft team to deliver the optimal results. It is also time-consuming regarding appliance fabrication and weekly adjustments. NAM therapy requires actively participating caregivers who are willing to commute for weekly appointments and routine care of the appliance. More importantly, NAM therapy requires healthy infants who can be seen on an out-patient basis and can tolerate having an intra-oral appliance. Infants who have problems with aspiration or other concerning medical conditions are not candidates for NAM therapy.

Provided herein is a novel nasal molding device having several advantages over prior devices. The novel nasal molding device improves nasal asymmetry in infants with CL/P who are medically compromised and cannot undergo traditional NAM therapy. A horizontal lip band component exerts force in concert with lip taping on a subject's protruding alveolar segment and indirectly molds the alveolar segments together. Implementing rigid material, the changes in a subject's anatomy is evident and defined. The device design does not interfere with feeding and allows proper force vectors, yet is simple to use and is well-tolerated by a subject. The device also requires minimal co-operation from caregivers as a treatment protocol requires less frequent adjustments. Taping does not require daily replacement but only when the tape loosens. The molding device is placed extraorally away from an oral cavity and avoids saliva contamination, and can be expected to stay on without replacement for at least 2-3 days.

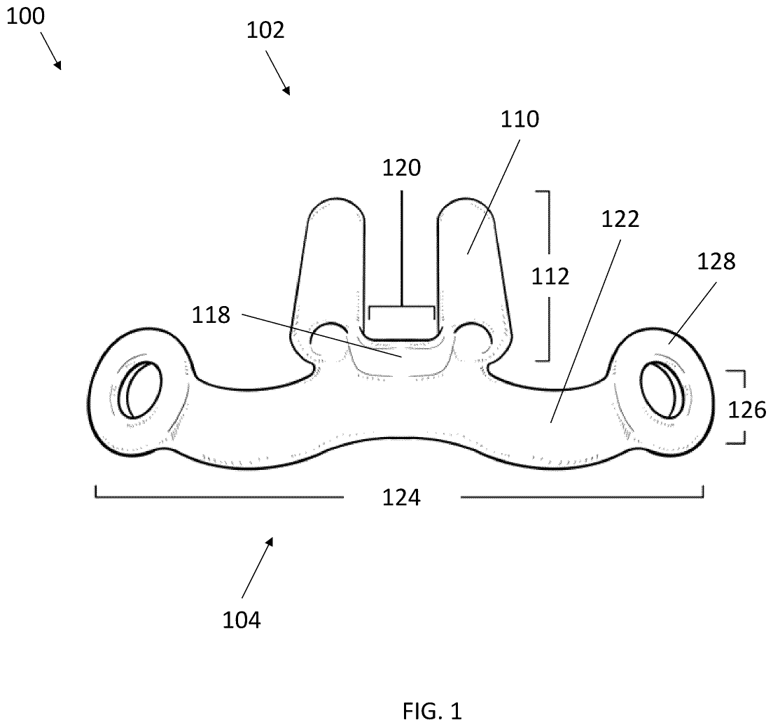

Referring now to FIG. 1 through FIG. 3Q, an exemplary nasal molding device 100 is depicted. Device 100 has a superior end 102, an inferior end 104, an anterior end 106, and a posterior end 108, and comprises at least one molding stent 110, a columella bridge 118, and a lip band 122. In certain instances, device 100 as described herein may be referred to as an approximator.

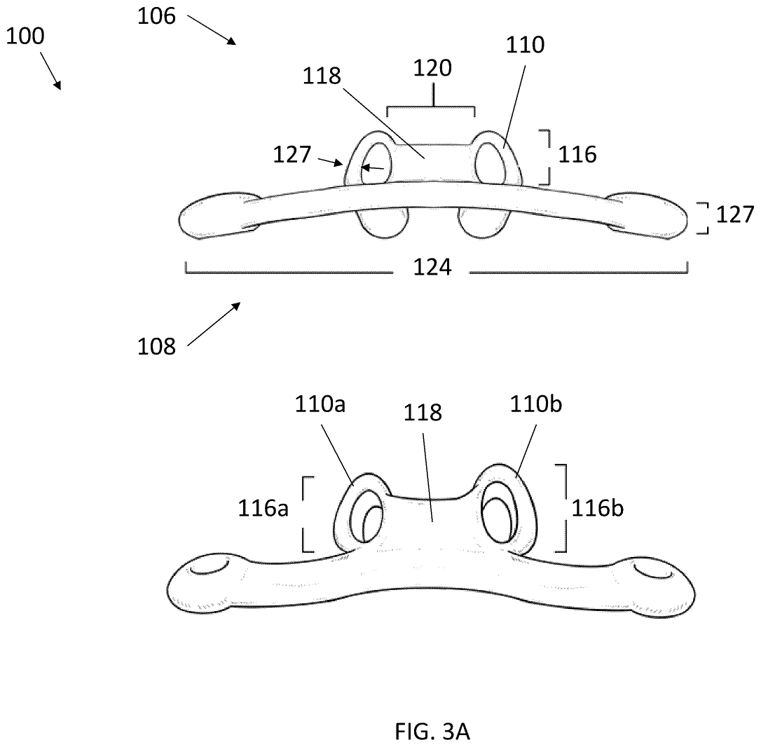

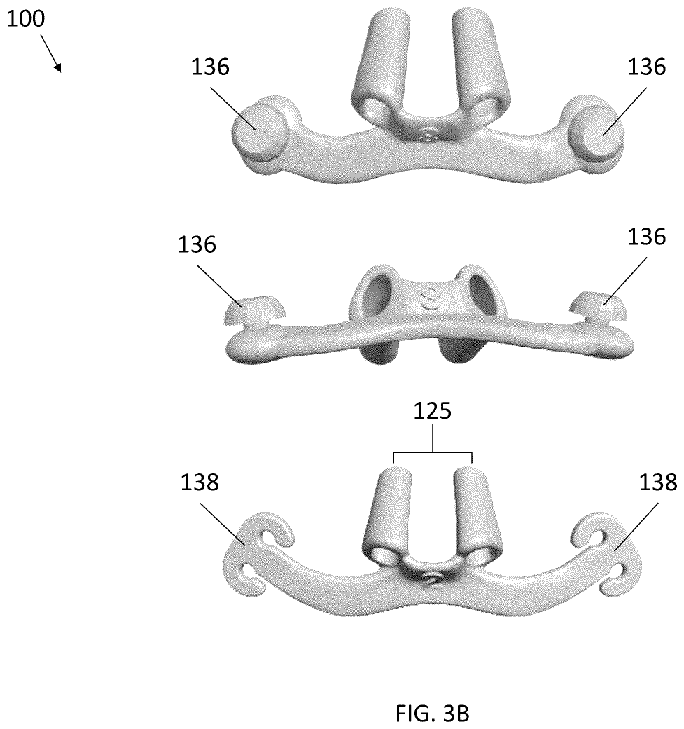

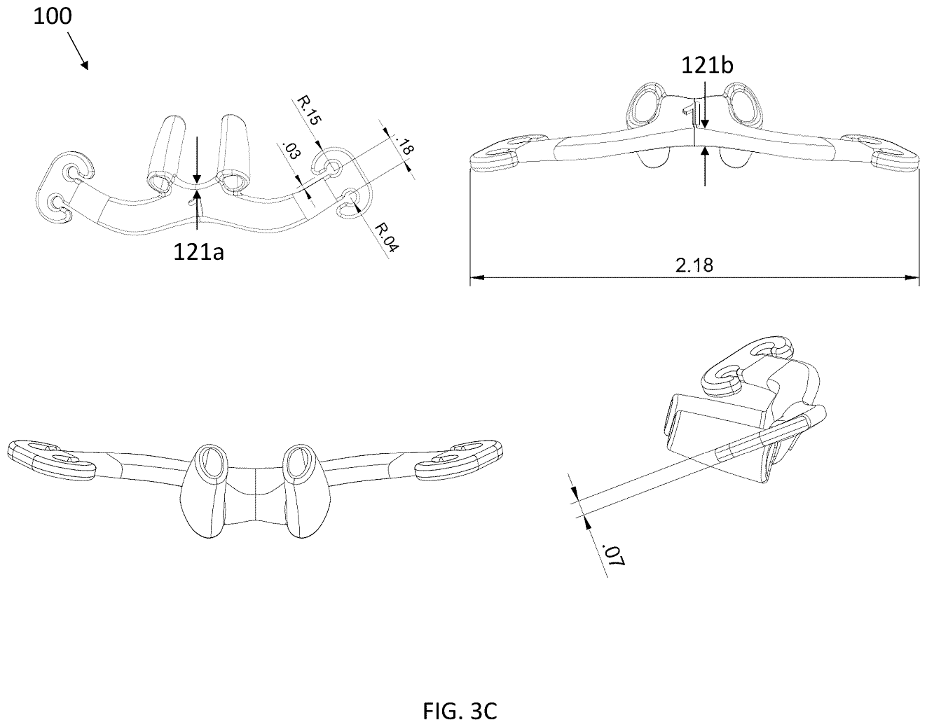

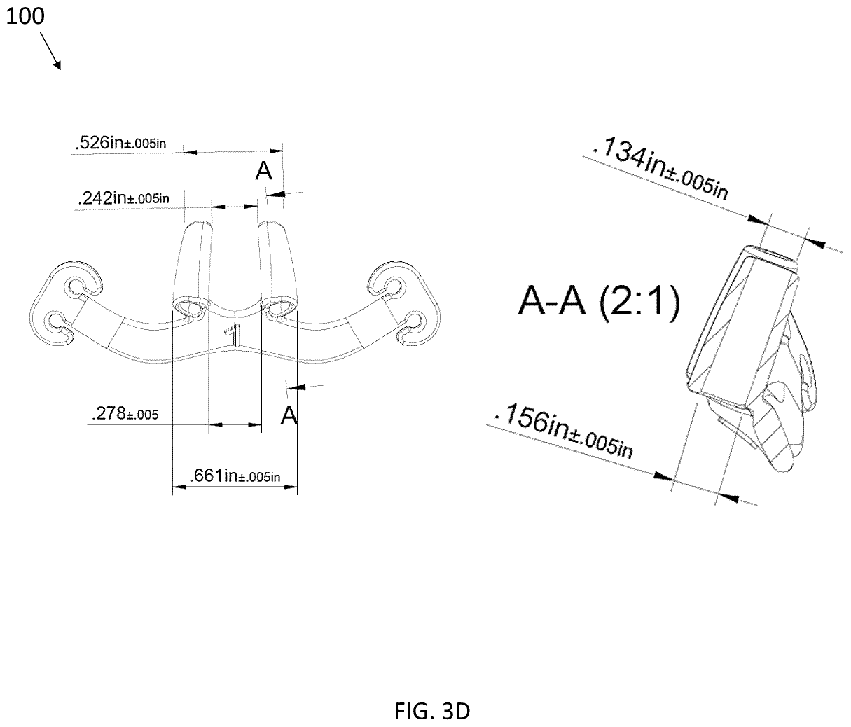

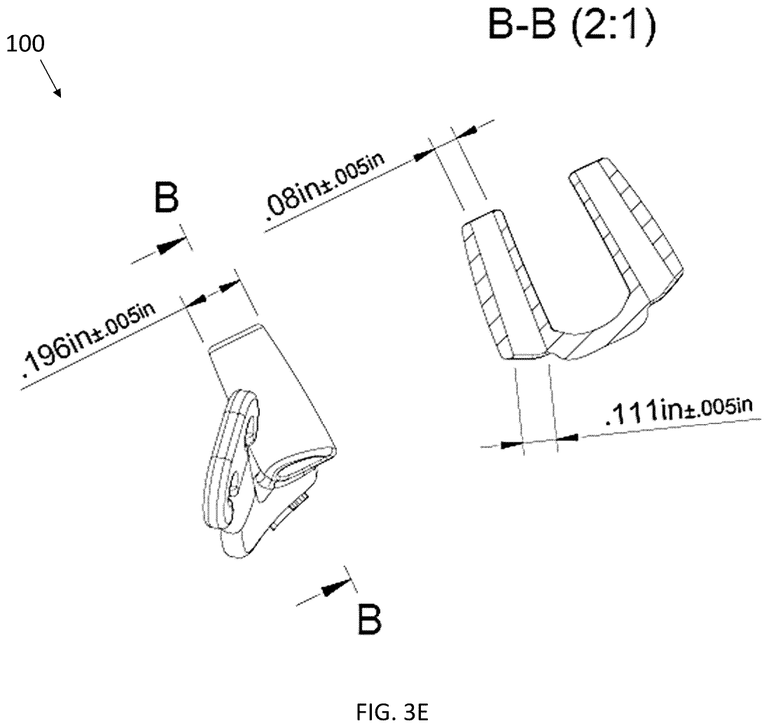

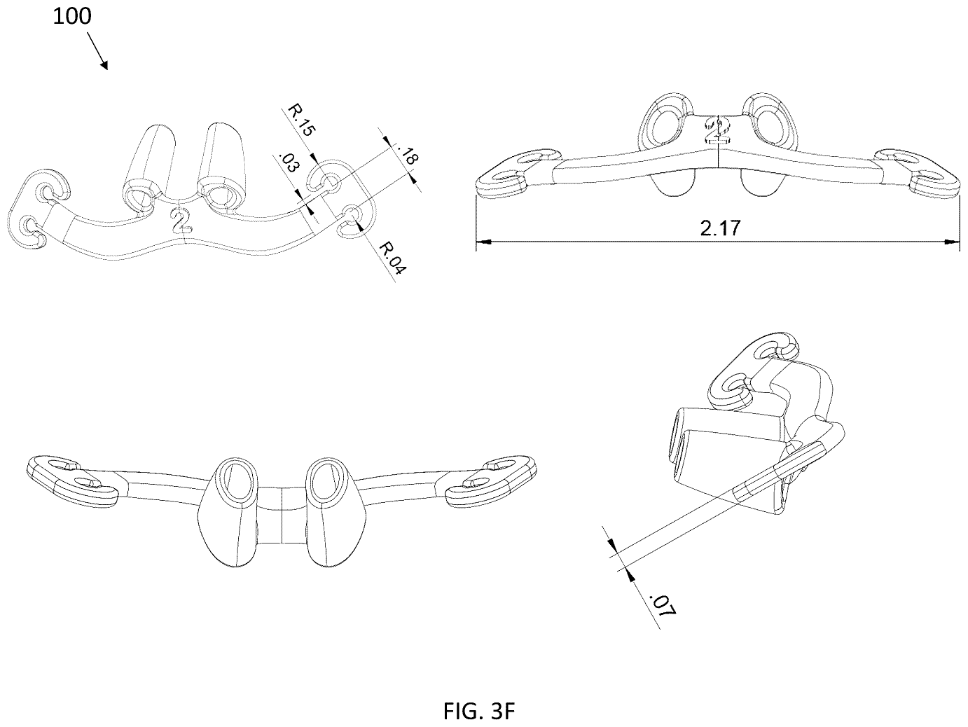

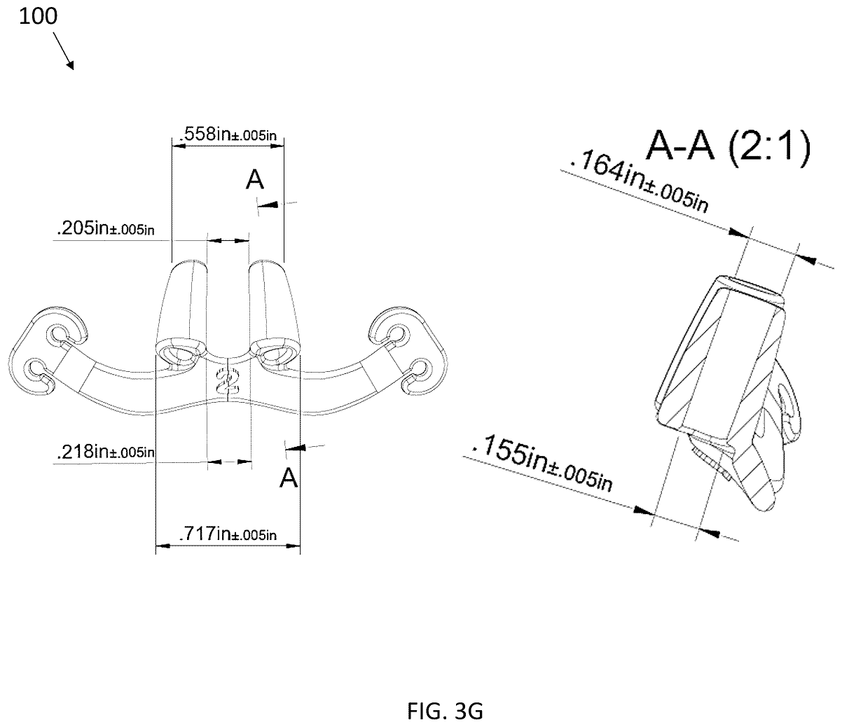

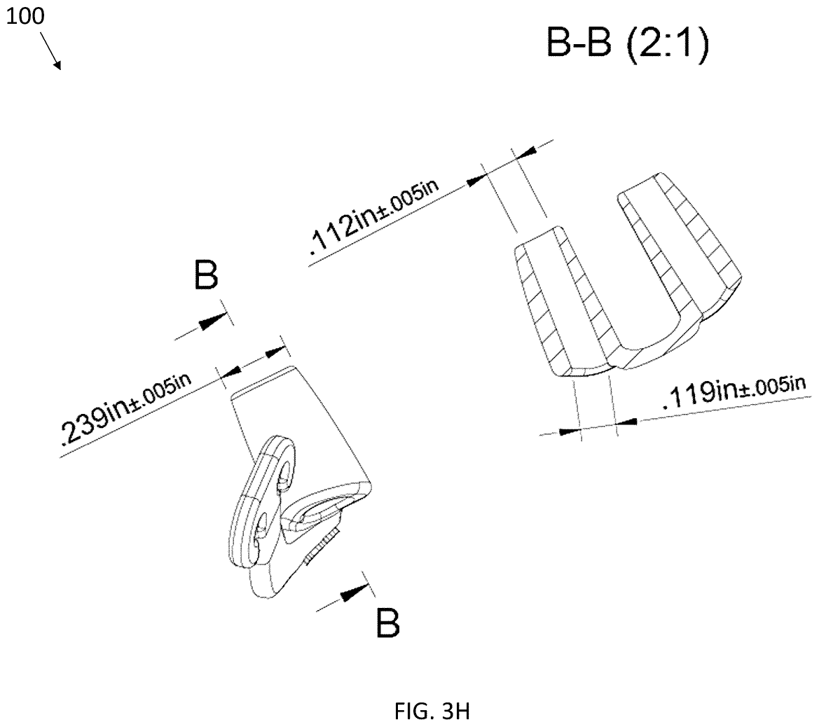

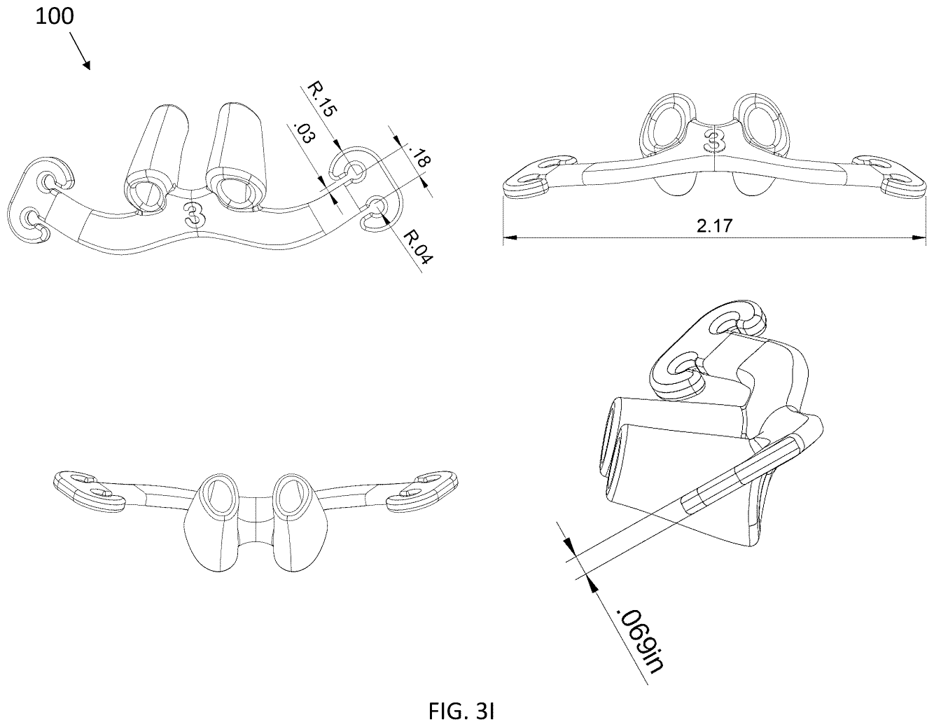

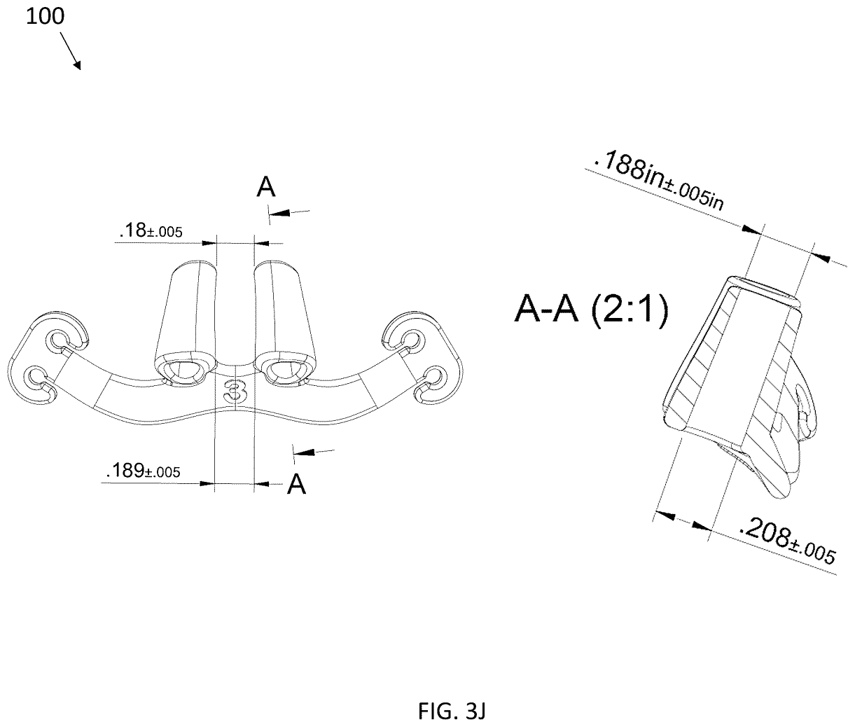

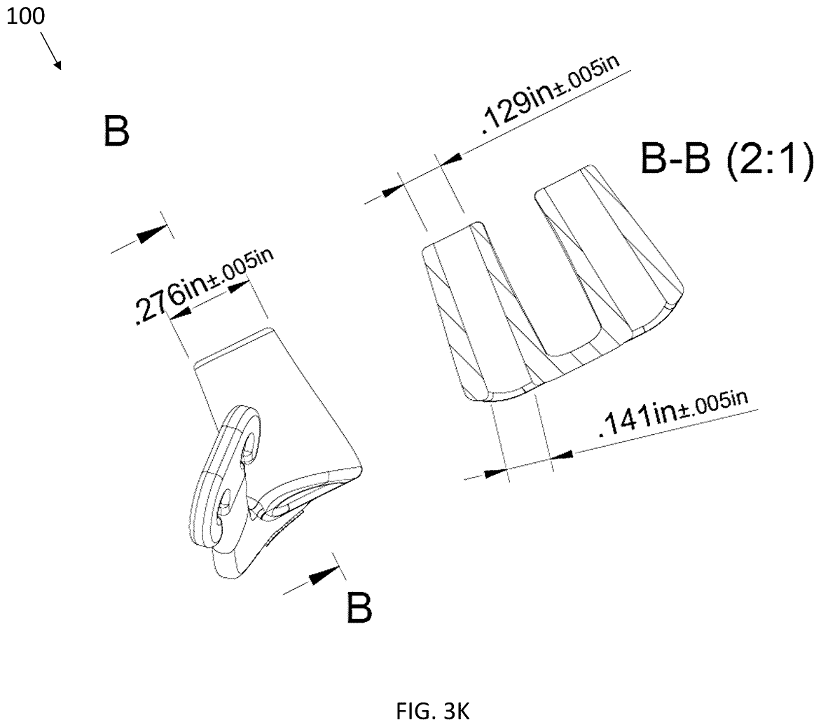

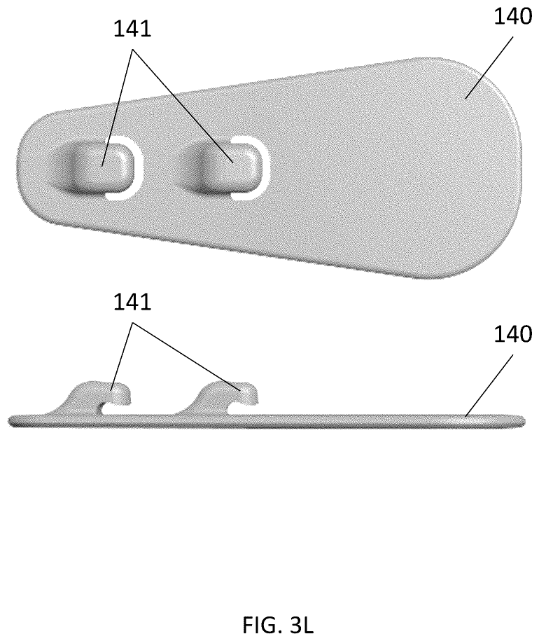

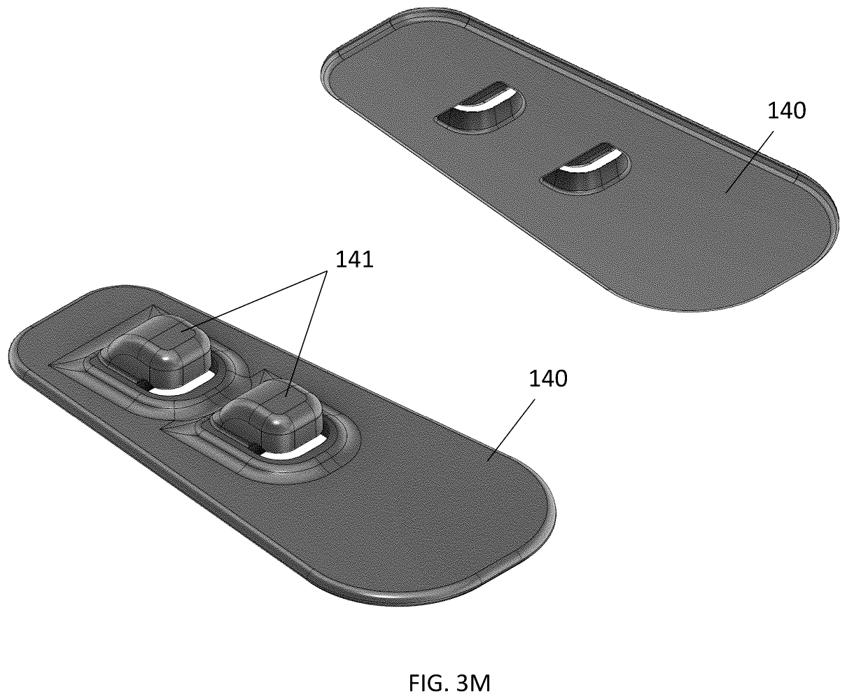

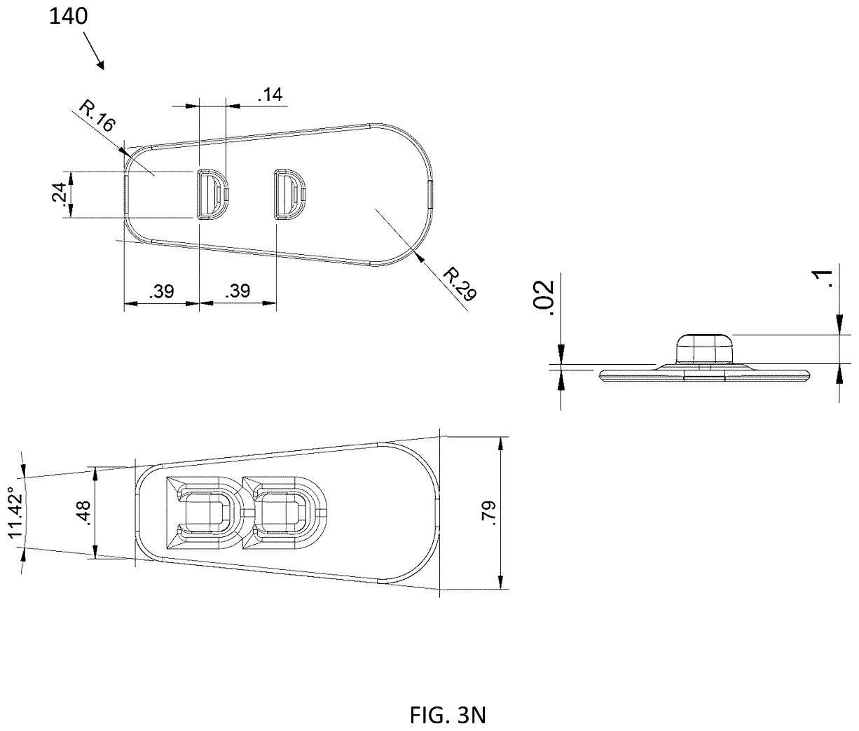

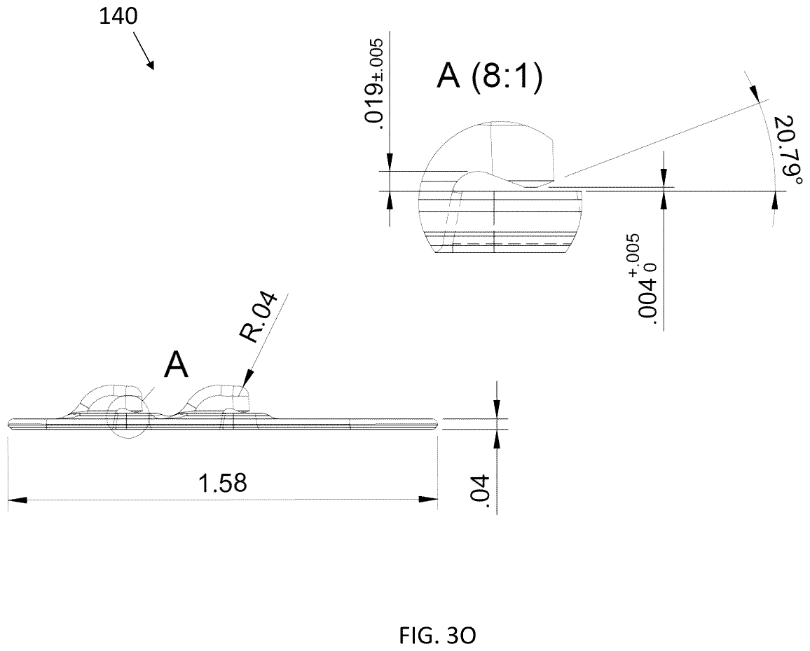

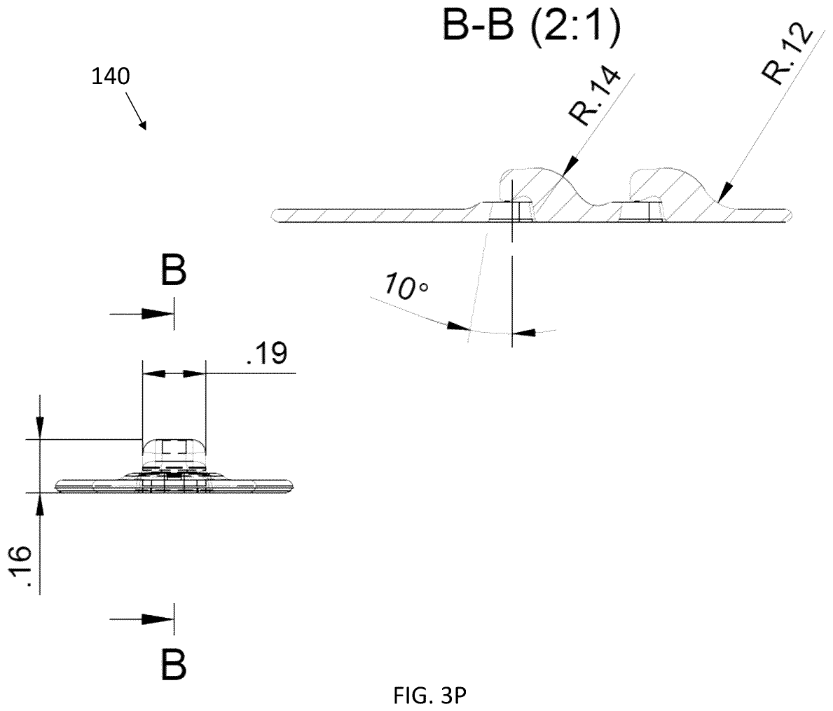

Molding stent 110 comprises a substantially tubular shape having a length 112 extending between a superior end having a superior width 114 and an inferior end having an inferior width 116. Molding stent 110 is configured to fit within a subject's nostril, such that lateral forces may be applied against an interior of the subject's nostrils through molding stent 110. Accordingly, molding stent 110 can comprise a hollow interior having at least one superior opening and at least one inferior opening, enabling unobstructed breathing when positioned within a subject's nostril. Molding stent 110 can have any suitable dimensions, such as a length 112 between about 5 and 20 mm (such as a length 112 between about 12 and 18 mm), a superior width 114 between about 1 and 15 mm, and an inferior width 116 between about 1 and 15 mm. In some embodiments, device 100 comprises two molding stents 110 having the same dimensions. In some embodiments, device 100 comprises two molding stents 110 having different dimensions. For example, as shown in the bottom image of FIG. 3A, a device 100 is depicted having a molding stent 110a and a molding stent 110b, wherein molding stent 110a comprises an inferior width 116a that is smaller than an inferior width 116b of molding stent 110b. In some embodiments, the superior ends of the molding stents 110 are separated by a distance 125.

In various embodiments, molding stents 110 can have a uniform material thickness or a varied material thickness, which affects the rigidity of molding stents 110. For example, in some embodiments, molding stents 110 can have a uniform rigidity along an entire length 112, while in other embodiments, molding stents 110 can have a greater thickness and rigidity at one end and a lesser thickness and rigidity at an opposing end. In some embodiments, molding stents 110 are more rigid at an inferior end and less rigid at a superior end, while in other embodiments, molding stents 110 are more rigid at a superior end and less rigid at an inferior end. Contemplated ranges for molding stent 110 rigidity can be described in terms of Shore Hardness, such as Shore Hardness between about between about 0 Shore OO and 100 Shore OO, or between about 50 Shore OO and 90 Shore OO.

In some embodiment, the at least one molding stent 110 comprises a wall thickness 127. In some embodiments, wall thickness 127 is variable along length 112 of molding stent 110. For example, in some embodiments, wall thickness 127 is thicker on the inferior end, and thinner on the superior end, providing a stiff inferior end, and a compliant superior end.

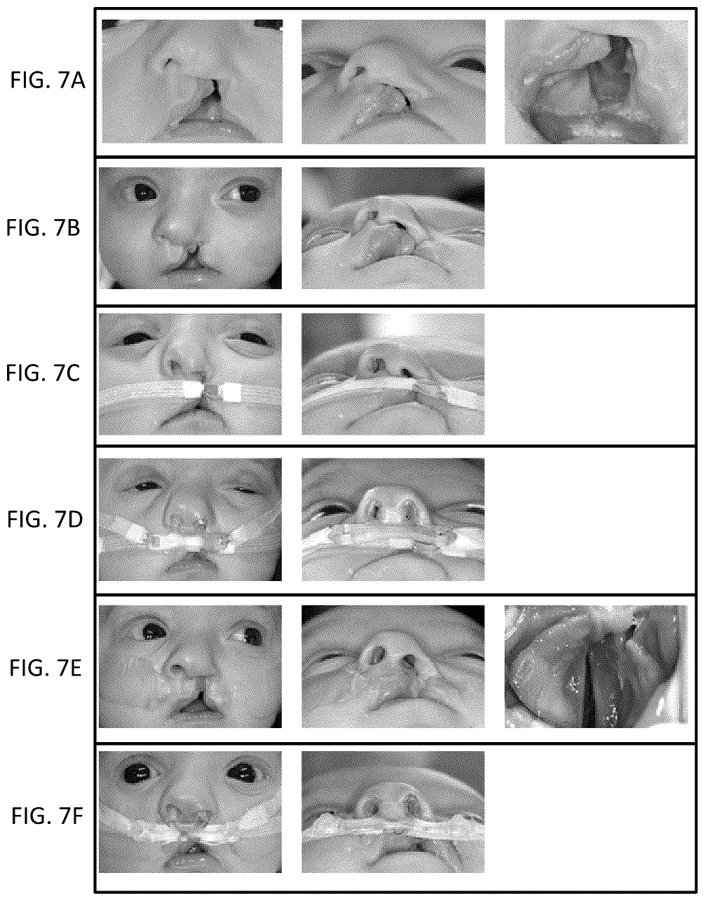

Each molding stent 110 is connected at an inferior end 104 to a columella bridge 118 and a lip band 122. Columella bridge 118 is positionable and/or disposed between adjacent molding stents 110, wherein a width 120 of columella bridge 118 defines a spacing between adjacent molding stents 110. Columella bridge 118 can have any suitable dimensions, such as a width 120 between about 1 and 15 mm and a height 121 between about 1 and 10 mm. In some embodiments, columella bridge 118 comprises a variable width 120 and/or height 121. In some embodiments, columella bridge 118 comprises a superior height 121a and an inferior height 121b. In some embodiments, superior height 121a is greater than inferior height 121b. In some embodiments, columella bridge 118 comprises a triangular cross-section, wherein superior height 121a is greater than inferior height 121b. In some embodiments, this variable height provides more rigidity to maintain the angle of molding stents 110 in an anterior direction. In some embodiments, columella bridge 118 comprises at least one surface, intended for contact with the skin of the subject, with a rounded surface and/or edge. Lip band 122 is positioned inferior to molding stent 110 and columella bridge 118 and extends in opposing lateral directions. Lip band 122 has a substantially planar shape and can have any suitable dimensions, such as a length 124 between about 20 and 100 mm, a height 126 between about 1 and 15 mm, and a thickness between about 0.5 mm and 5 mm. Lip band 122 can have any desired shape. For example, lip band 122 can have a substantially linear construction along length 124, or comprise one or more localized curvatures and/or bends for enhanced fitment to a subject's upper lip. A first curvature can be positioned inferior to columella bridge 118 and conform to a subject's cupid's bow. The first curvature also facilitates feeding. A second and third curvature can be positioned laterally and adjacent to columella bridge 118 and conform to a subject's alar base. Lip band 122 can further curve in a posterior direction, as depicted in FIG. 3A (top), and conform to the curvature of a subject's philtral ridges and upper lip. The posterior curvature can be between about 10° to about 45°, such as an angle of about 30°. For example, in some embodiments, lip band 122 comprises at least one surface, intended for contact with the skin of the subject, with a rounded surface and/or edge. In some embodiments, lip band 122 comprises opposing lateral ends having a loop connector 128. Loop connector 128 is configured to receive one or more soft elastic straps or bands (visible in FIG. 3Q, FIG. 7D, FIG. 7F) securing device 100 to a subject. In some embodiments, lip band 122 comprises opposing lateral ends having a button connector 136 configured to receive one or more loops of soft elastic straps or bands (visible in FIG. 3B top and middle). In some embodiments, lip band 122 comprises opposing lateral ends having a hooked connector 138 configured to receive one or more loops of soft elastic straps or bands (visible in FIG. 3B, bottom). In some embodiments, hook connector 138 is configured in the shape of an anchor. In some embodiments, hook connector 138 has rounded surfaces and/or rounded edges. In some embodiments, the tines of hook connector 138 are angled out in a posterior direction to enable easy attachment and detachment of an elastic loop or band. In some embodiments, hook connector 138 is configured to easily engage elastic and remove elastic without having to remove device 100, retainers 140 and/or adhesives from the skin of the subject, and helps to prevent skin irritation. It should be understood that device 100 is not limited to the depicted connectors, and that any connector configured to receive one or more soft elastic straps or bands are contemplated.

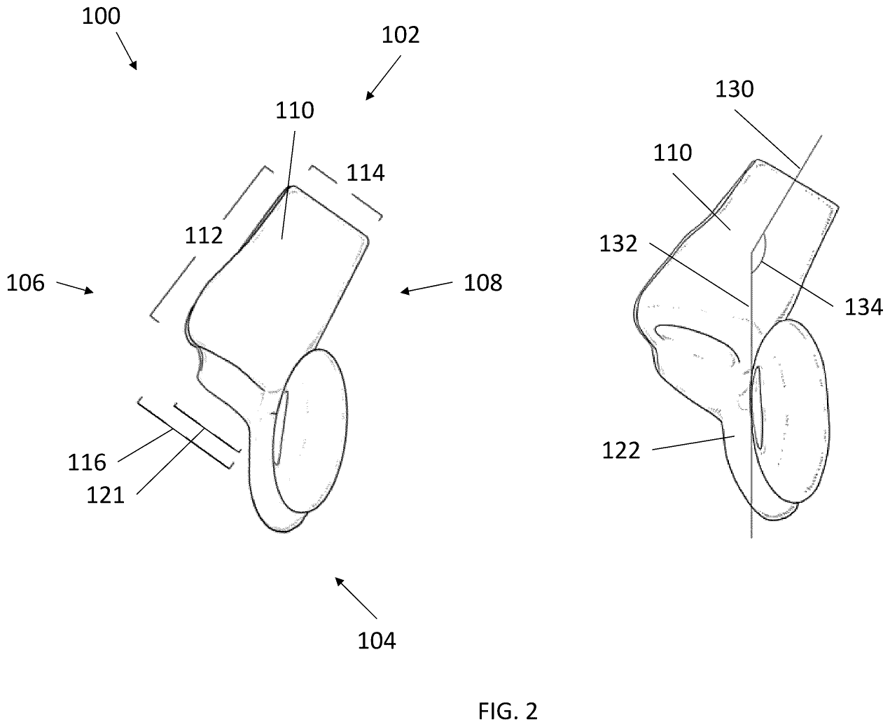

In some embodiments, molding stents 110 and lip band 122 are aligned at a specific angle. For example, visible in the right image of FIG. 2, molding stent 110 can comprise a molding stent axis 130 aligned in parallel with length 112 of molding stent 110, and lip band 122 can comprise a lip band axis 132 aligned in parallel with height 126 of lip band 122. Angulation 134 is formed at an intersection between molding stent axis 130 and lip axis 132 and can be used to characterize the alignment between molding stents 110 and lip band 122. In various embodiments, angulation 134 can be between about 90° and 180°, between about 110° and 160°, or between about 120° and 150°, such as an angle of about 144°. In some embodiments, device 100 comprises two molding stents 110 having the same angulation 134. In some embodiments, device 100 comprises two molding stents 110 having different angulations 134.

While exemplary nasal molding devices of the present invention are described above, the molding devices are nonetheless amenable to any suitable modification to augment their function. For example, in certain embodiments, the molding devices further comprise one or more retainers 140 shown in FIG. 3Q. In certain instances, retainers 140 as described herein may be referred to as fasteners. Each of retainers 140 comprise a planar body having a top and a bottom surface, wherein the top surface comprises one or more connectors configured to receive one or more soft elastic straps or bands 142 connected to a molding device (as demonstrated in FIG. 3Q). Each of retainers 140 can have any desired dimensions configured to fit the one or more connectors, such as a length and a width each between about 10 mm and 100 mm. For example, retainers 140 are depicted in FIG. 3L and FIG. 3M as having hooked connectors 141 placed at two positions such that one or more elastic straps or bands 142 can be adjusted between the two positions. Contemplated connectors for retainers 140 include but are not limited to the loop connectors 128 of device 100, button connectors 136 of device 100, hook connectors 138 of device 100, hook connector 141, hook and loop fasteners, clips, magnets, and the like. The bottom surface of each of retainers 140 is configured to adhere to a subject's face or head (such as the cheek or temple), such as with an adhesive, a glue, or a tape. In some embodiments, the inner surface of each of retainers 140 is provided with a non-irritating interface, such as a hydrocolloid tape or adhesive. For example, the non-irritating interface can be placed on the subject's face or head such that retainers 140 are re-positionable on the interface surface as needed without further irritation of the subject's underlying skin. In various embodiments, retainers 140 are configured to adhere to a subject's face or head for a period of 1-2 weeks. In some embodiments, retainers 140 secure device 100 and/or at least one molding stent 110 to the nose of the subject without the use of additional tapes or adhesives applied to device 100.

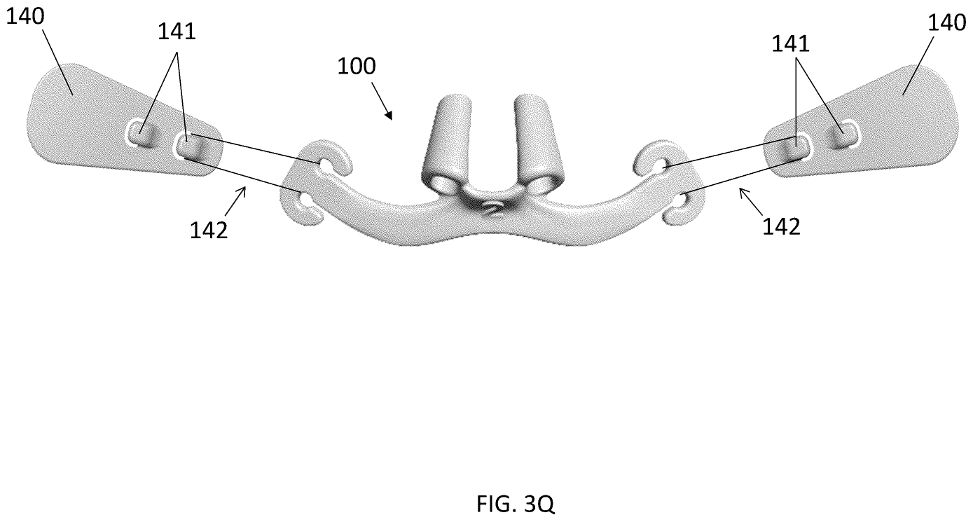

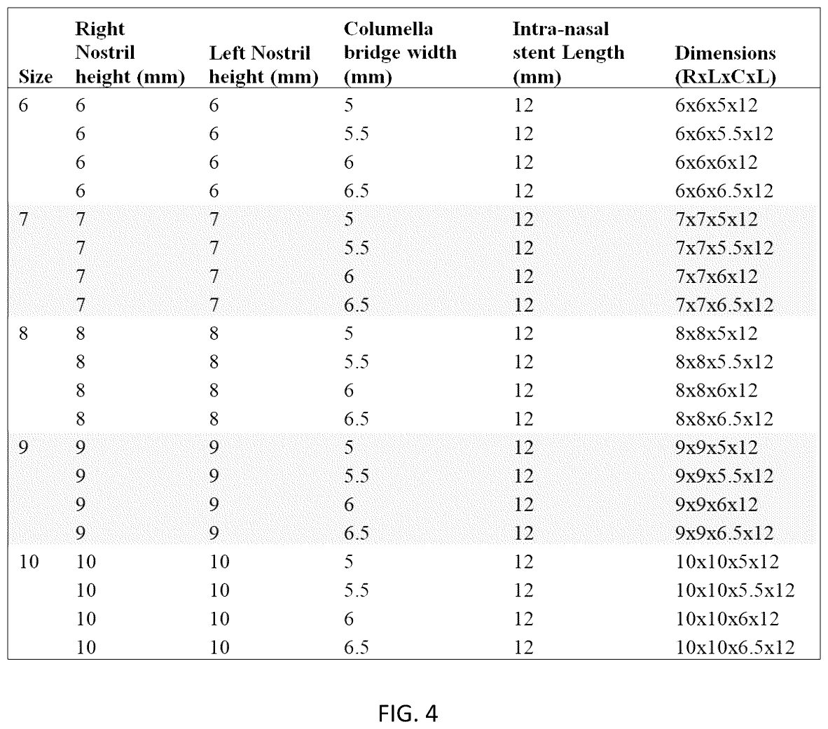

In another example, in certain embodiments, the molding devices are fabricated with a monolithic structure, such that the molding stents, columella bridge, and lip band are formed from a single continuous piece of material. Monolithic molding devices can be made to order based on a subject's anatomy, or provided in preselected sizes, such as in the table shown in FIG. 4. Non-limiting examples of pre-selected size include: molding stent widths (depicted as right and left nostril heights) between about 6 mm and 10 mm in 1 mm increments; columella bridge widths between about 5 mm and 6.5 mm in 0.5 mm increments; and molding stent lengths of about 12 mm. A pre-selected size be identified by a label displayed on a molding device (such as the number “8” and the number “2” depicted in FIG. 3B). Contemplated sizes can be numbered sequentially or non-sequentially, including but not limited to sizes 1, 2, 3, 4, 5, and the like, or sizes extra small, small, medium, large, extra large, and the like. Further embodiments of contemplated sizes (i.e. size #1, size #2, and size #3) with dimensions may be found in FIG. 3C through FIG. 3K.

In some embodiments, as molding stent 110 increases in size (e.g. from a size #1 to a size #3), distance 125 is decreased. In some embodiments, distance 125 is decreased as the size of molding stent 110 is increased in order to help approximate the nasal cartilage medially. In some embodiments, as the at least one molding stent 110 increases in size (e.g. from size #1, to size #2, to size #3), width 120 of columella bridge 118 is reduced accordingly. In some embodiments, this reduction of width 120 helps to make the nose narrower as the subject gets older, and also helps to make the columella of the subject longer. In some embodiments, the wall thickness 127 of the at least one molding stent 110 increases in stiffness and/or firmness (e.g. soft, medium, hard) as the size of the at least one molding stent 110 increases in size (e.g. from size #1, to size #2, to size #3). In some embodiments, the increase in stiffness as the size increases helps mold the nasal cartilage of the subject. For example, in some embodiments, molding stent 110 with a size #1 is soft during the first few weeks of birth of the subject, and the size and firmness is increased progressively.

In certain embodiments, the molding devices can be fabricated with a modular structure, such that one or more of the molding stents, columella bridge, and lip band can be fabricated separately in different sizes, selected based on a subject's anatomy, and assembled together to form a completed nasal molding device. The assembly means can include but are not limited to snap-fitting components, gluing components, or bonding components.

The nasal molding devices of the present invention can be made using any suitable method known in the art. The method of making may vary depending on the materials used. For example, components substantially comprising a metal may be milled from a larger block of metal or may be cast from molten metal. Likewise, components substantially comprising a plastic or polymer may be milled from a larger block, cast, or injection molded. In some embodiments, the components may be made using 3D printing or other additive manufacturing techniques commonly used in the art. Contemplated materials include but are not limited to acrylic, plastic, and silicone. In some embodiments, the materials can withstand commonly used sterilization techniques, enabling the devices to be reusable. In some embodiments, the materials are subject to post-processing steps to enhance biocompatibility and optimize mechanical properties. Contemplated post-processing steps include but are not limited to: rinsing (such as in an alcohol or water bath); post-curing (such as exposure to ultraviolet light or in a heated environment); and trimming (such as with a rotary tool and bur). In other embodiments, inexpensive methods permit the devices to be single-use and disposable. In various embodiments, the components may include a pliable material, such that adjustments can be made to the device after fabrication.

### Methods of Cleft Lip and Palate Repair

The present invention also relates to methods of repairing cleft lip and palate using the molding devices described herein. The methods are effective in minimizing deformities associated with cleft lip and palate in preparation for primary surgical repair, such that predictable and esthetic outcomes are achievable. In some embodiments, the methods are effective in improving the position of a subject's alveolus in reducing an alveolar cleft defect size. In some embodiments, the methods are effective for treating incomplete and/or complete cleft lip and/or cleft palate. In some embodiments, the methods described herein can be referred to as a Presurgical Lip, Alveolus, and Nose Approximation (PLANA).

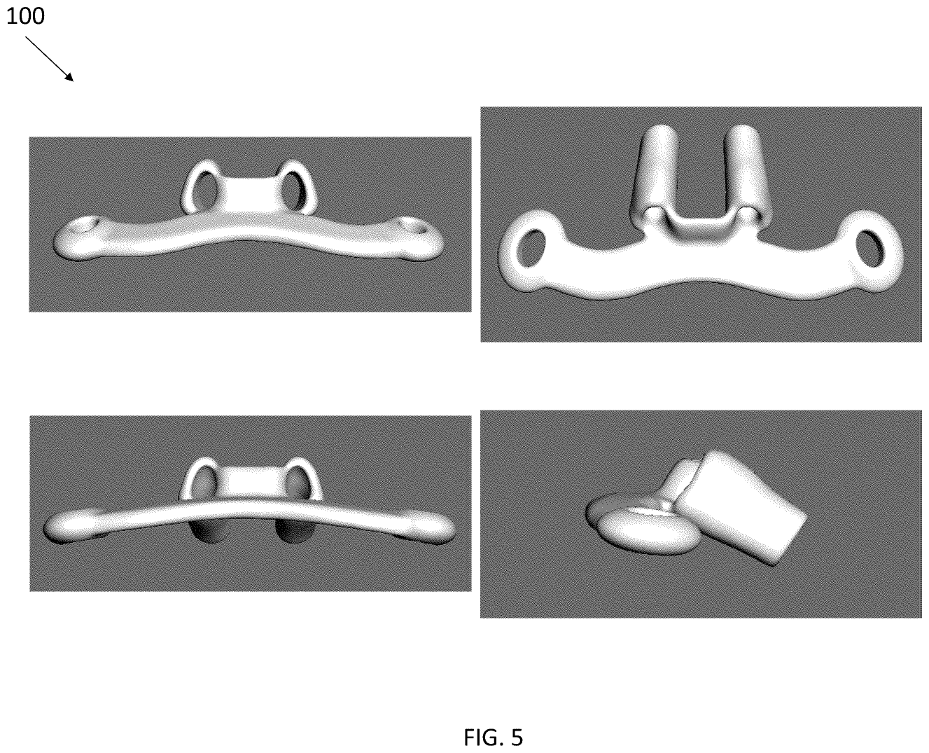

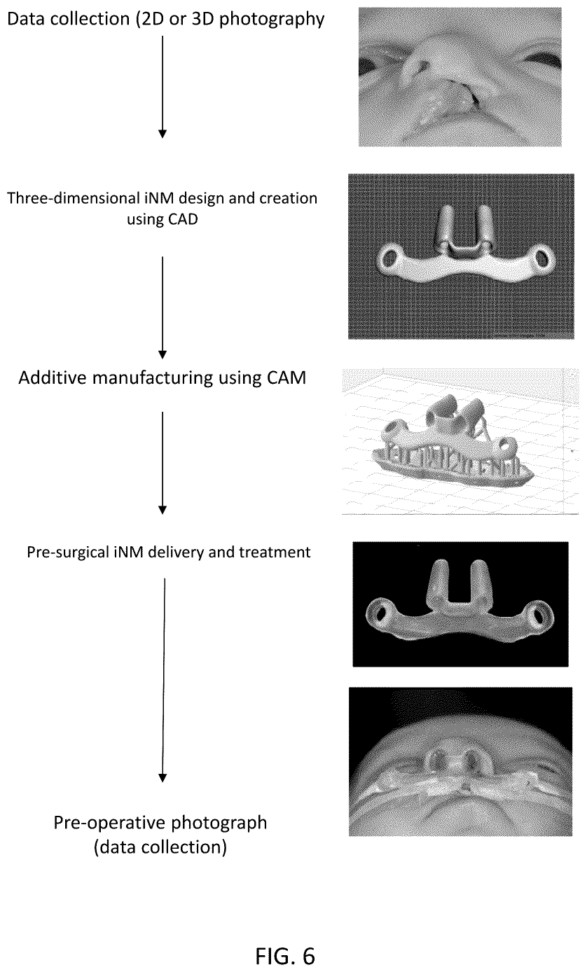

In some embodiments, the methods begin with fabricating a molding device for a patient. In some embodiments, the molding device can be fabricated using a workflow depicted in FIG. 6, wherein a 3D model of a nasal molding device is created (FIG. 5). The dimensions and orientation of the molding device can be based on 3D scans of a subject's face or from reconstructions based on 2D images of a subject's face.

In some embodiments, the methods begin with the provision of a selection of molding devices in preset sizes and the selection of an appropriately sized molding device for a subject. The selection of molding devices can have any suitable size range, such as in the table depicted in FIG. 4. Contemplated sizes can be numbered sequentially or non-sequentially, including but not limited to sizes 1, 2, 3, 4, 5, 6, 7, 8, 9, 10, and the like. In some embodiments, a size number references a width or height of the left and right molding stents with varying columella bridge widths. The preset sizes can also include asymmetrical molding devices, wherein a first molding stent has a size that is different from a second molding stent. In certain embodiments, the selection of molding devices can include modular components. As described elsewhere herein, a modular molding device can include separable molding stents, columella bridge, and lip bands that are fabricated separately in different sizes. Accordingly, the preset sizes can include molding stents in different sizes, columella bridges in different widths, and lip bands in different lengths. Individual components may be selected based on a subject's anatomy, and the selected components assembled together to form a completed nasal molding device.

Once an appropriately sized molding device is prepared, the device can be introduced to a subject by inserting the molding stents into a subject's nostrils and securing the lip band to the subject's upper lip and face such that force vectors are applied on the nostrils and face as appropriate. The device can be secured in any desired fashion, including but not limited to the application of a taped adhesive on the lip band and the subject's face, the application of an adhesive (such as a medical grade adhesive) between the lip band and the subject's face, the use of elastic straps or bands attached to connectors on the lip band and secured to the subject's face or head, as well as the use of elastic straps or bands attached between connectors on the lip band and connectors on device retainers adhered to the subject's face or head.

In some embodiments, the securing of the device to a subject can be applied asymmetrically or at an angle to apply the appropriate force vectors. In some embodiments, between about 0 g and 125 g of force, such as about 100 g of force, is effective in reshaping a subject's nose, aligning the lips, and molding the gums. In some embodiments, the amount of force is decreased over time or with the application of a successively sized device. For example, taping or adhesives can be applied such that the device is angled upwards to maintain the retention of the device in the nostrils, and asymmetrical tension can be biased to oppose the direction of a nasal deformity and/or cleft to encourage up-righting of the columella, aligning the nasal structure to midline, and closure of cleft gaps. In some embodiments, the positioning of the device can be altered over time to adjust for the gradual improvement of nasal alignment and cleft closure, or the device can be replaced with a new device that is more appropriately sized for the subject's adjusted anatomy. For example, a treatment regimen can progress from a first device size to a second or more device sizes, wherein each successively sized device is each worn for a treatment period of about 1-4 weeks for a total treatment period of about 10-12 weeks. In some embodiments, the methods may comprise choosing a device size and/or treatment duration based on the age of the subject. In some embodiments, the methods may comprise changing the device and/or changing the size of the device as the age of the subject increases. In some embodiments, the methods may comprise switching the device for a larger sized device as the subject gets older (e.g. size #1 for a first duration, size #2 for a second duration, size #3 for a third duration, etc). In some embodiments, the methods may comprise changing the elastic bands, increasing the strength of the elastic bands, and/or changing the position of the elastic bands or retainers as the subject ages. In some embodiments, the methods are configured to prepare a subject for surgery, such that a treatment regimen is applied pre-surgery. In some embodiments, the methods are configured to rehabilitate a subject after surgery (such as to prevent relapse of the nose), such that a treatment regimen is applied post-surgery. In some embodiments, the methods may comprise a silicone cleft nasal approximation device 100 that provides gentle support to the soft tissue of the nose and approximates the displaced soft tissue of the nose, lip and alveolus. In some embodiments, the methods may include the use of device 100 for treatment and/or supporting a collapsed upper and lower nasal cartilages, treatment and/or supporting a displaced columella of the nose, treatment and/or supporting a deviated nasal septum, and treatment and/or approximating a nasal alar bases and/or alveolar segments.

### Kits

The present invention also provides kits for nasal molding. The kits include the molding devices described elsewhere herein, as well as relevant accessories and instrumentation. For example, in some embodiments, the kit can comprise a series of molding devices in a range of sizes, as well as retainers, adhesives, tapes, glues, elastic straps, and elastic bands.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, utilize the present invention and practice the claimed methods. The following working examples therefore, specifically point out exemplary embodiments of the present invention, and are not to be construed as limiting in any way the remainder of the disclosure.

### Example 1: Pre-Surgical Individual Nasal Molding for Infants with Cleft Lip and/or Palate Using CAD/CAM Technology

The following study documents the innovative use of CAD/CAM technology to aid in design and fabrication of a pre-surgical individual nasal molding (iNM) device in an infant with complete unilateral cleft lip and/or palate (CLP).

### Case Description

A 4-week-old non-hispanic white male infant with complete unilateral CL/P was assessed for NasoAlveolar molding (NAM) therapy. In addition, the patient had diagnoses of chromosome 4P deletion, horseshoe kidney, agenesis of corpus callosum, heart murmur, and difficulty in feeding. Patient was put in the Neonatal Intensive Care Unit for 4 weeks after delivery. Due to these several underlying medical conditions, it was decided that the patient was not a good candidate for NAM therapy. However, this patient's family was offered to start with lip taping for few weeks and then started with a customized 3D-printed iNM device to mold the nasal cartilages.

The materials and methods are now described.

Designing Platform and the Concept of the Design and Dimension

Autodesk Meshmixer (Autodesk Inc., San Rafael, CA) was used to construct a 3-dimensional (3D) pre-surgical iNM device. First, a 3D facial scan of a patient (.OBJ format) was uploaded into the program Autodesk Meshmixer. The 3D construction of the pre-surgical iNM model was performed using a generic age-matched infant's facial scan as a dimensional guide. After the 3D model construction was completed, the file was exported as a stereolithography (.STL) file.

The iNM device has 3 main components: the bilateral intra-nasal molding stents, the columella bridge, and the horizontal acrylic lip band. The stents are ovoid in shape similar to the nostril anatomy and tapered in length with the end diameter height 2 mm smaller than the nostril diameter height. Standard iNM devices can be designed in different sizes, the smallest diameter height started at 7 mm and each size was 1 mm incrementally bigger. Both sides of the stents are connected with the columella bridge that is 5 mm in width and 1 mm thick. The columella bridge length was designed in 4 sizes (5 mm, 5.5 mm, 6 mm, and 6.5 mm) which follow Farkas's one-year-old infants' published norm for columella width (Farkas L G. Anthropometry of the Head and Neck, 2nd Edition. New York: Raven Press; 1994. p. 427). The appropriate columella bridge size for each infant can be chosen based on clinical exams. The nasal stents and columella bridge connect to the 3^{rd} part of the iNM, the horizontal acrylic lip band, at a 30° angle in a downward direction. The horizontal acrylic lip band was designed to enhance an effect of passive alveolar molding from lip taping. It has a wave-like design comprises of 3 parts: the middle part, the lateral parts, and the elastic loops. The middle curvature was designed to accommodate the bottle nipple during feeding and the lateral parts with elastic loops curve 30° upwards, backwards, and laterally intended for an optimal adaptation to the face and allow proper vector of elastic application for retention. The dimension of the horizontal acrylic lip band is 45 mm long, 5 mm wide, and 1 mm thick. An exemplary design of the iNM device is illustrated in FIG. 1.

Printer and Material for Printing

The pre-surgical iNM device was printed using a commercially available resin-based stereolithography apparatus (SLA) desktop 3D printer (Form 2, Dental LT Clear Resin, Formlabs; Somerville, MA) via PreForm software. The Dental LT Clear resin is a biocompatible photopolymer resin and is classified as Class IIa CE-certified. The printing resolution for the pre-surgical iNM was 100 microns (0.1 mm).

Processing the Finished Product

The resin-based 3D printed iNM were post-processed in accordance with the manufacturer's recommendations using a 2-step process: rinsing and post-curing. First, the printed part was washed in isopropyl alcohol (96%) for 2 minutes, then transferred to a new bath of isopropyl alcohol (96%) for an additional 3 minutes. The printed part was allowed to dry thoroughly and inspected for any uncured resin before proceeding to the post-curing process. The post-curing step ensures biocompatibility and optimal mechanical properties. The tested exposure time of the printed part was 10 minutes to 108 watts each of Blue UV-A (315-400 nm) and UV-Blue (400-550 nm) light, in a heated environment at 60° C. (140° F.). Here, a TRIAD® 2000 Light Curing Unit (DENTSPLY International Inc., York, PA) was used, the unit having a halogen light source to cure the part with the use of heat and an intense blue light wavelength (400-500 nm) for 10 minutes. FIG. 6 illustrates a semi-digital workflow for iNM device construction.

Pre-Surgical Individual Nasal Molding (iNM) Therapy

Patient was started with lip taping using medical grade adhesive bandage (3M™ Steri-Strip™ Reinforced Adhesive Skin Closures) and the family was instructed to change the tapes daily. Medical grade adhesive (Mastisol® Liquid Adhesive) was recommended to reinforce the tapes if needed. Patient's family demonstrated good compliance with taping and noticeable changes in the lips as well as greater alveolar segment were observed. Three weeks into treatment as the lips and nose structures were improving, and it was reported that the patient was having difficulty in breathing with tight lip tapes. Patient was then planned to start with custom printed nasal stents to open the nasal passage as well as actively mold the nasal cartilages. The iNM treatment was initiated in concurrence with the horizontal lip taping. The 3D printed iNM device was then inserted in the nose with petroleum jelly as lubricant. The iNM device was secured to the cheek bones with orthodontic elastics and surgical tapes on both sides. The tapes were placed at a 20°-30° angle upward to maintain the retention of the iNM device in the nose. Family was encouraged to feed the patient to allow acclimatization to the new appliance. Family was informed to replace the tapes when they become loose. Since the iNM device was placed extra-orally away from any oral cavity and saliva contamination, it was expected to stay on without replacement for at least 2-3 days.

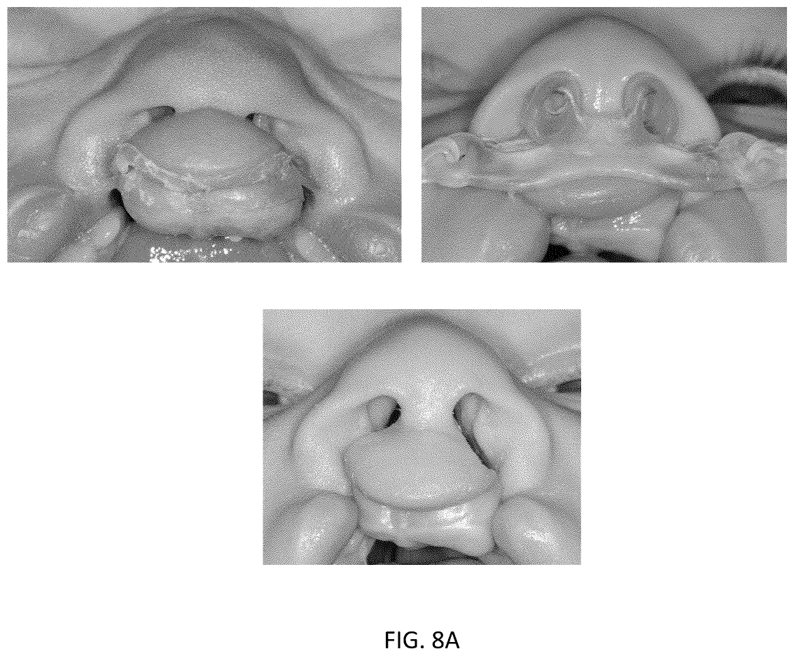

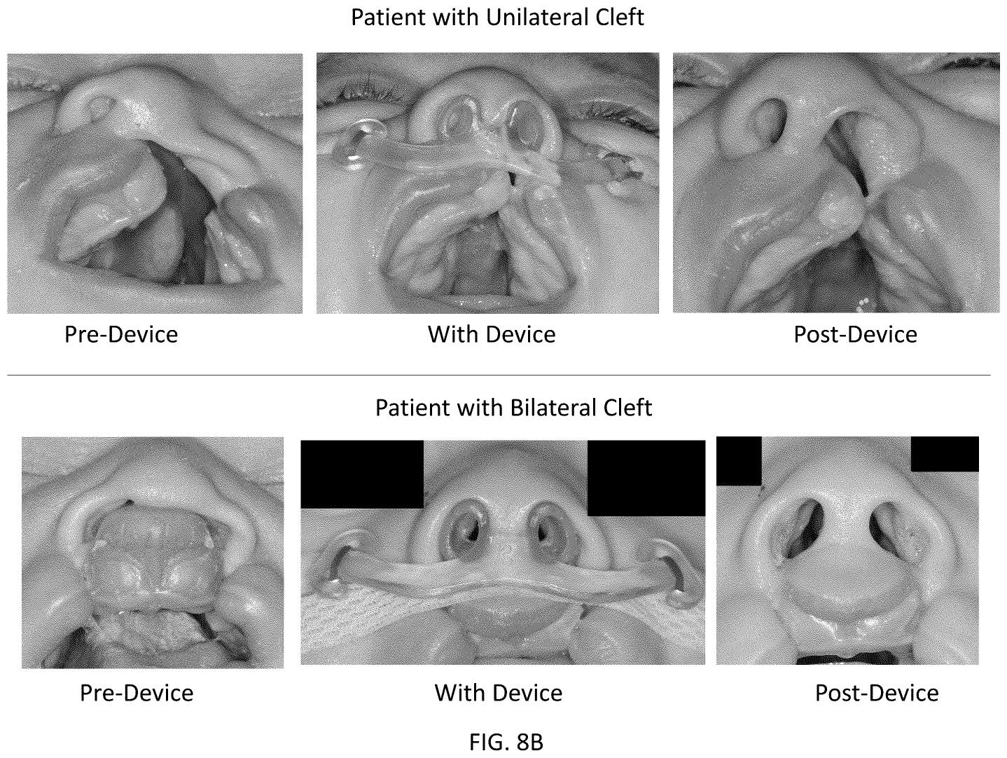

The patient was seen for the following week after delivery to check for any sores or irritation, then every 2 weeks for follow-up care. Once the iNM device became passive, it was replaced with a larger size iNM device to allow further nasal cartilage molding and up-righting of the columella. Asymmetrical elastic tension in the tapes was recommended towards the cleft side to bring the nasal structure to the midline. The improvement in nasal symmetry can be observed within the first week of the treatment. FIG. 7 demonstrates treatment progress using pre-surgical iNM device (FIG. 8 demonstrates treatment progress in a patient having a bilateral cleft lip and palate).

The results are now described.

Following 10 days after device delivery, the nasal asymmetry improved significantly FIG. 7E and cleft width of the alveolus reduced considerably. Asymmetric pre-surgical iNM was fabricated with 1 mm overcorrection in nostril height for the left cleft side nostril. No complications were reported during the course of treatment. The total number of treatment visits for the pre-surgical iNM was 6 appointments. Due to a rigid nature of the resin material, the intra-nasal molding part can impinge on nasal soft tissues, however this not reported with the patient. This problem can be easily addressed before the appliance delivery by trimming acrylic with a laboratory bur.

Pre-surgical Infant Orthopedics (PSIO) is offered by several multidisciplinary cleft centers around the world. A recent ACPA survey reported that 88% of the 82 cleft and craniofacial multidisciplinary programs were offering NAM as the appliance of choice for PSIO therapy (Khavanin N et al., The Cleft Palate-Craniofacial Journal. 2019 August; 56(7):860-6). Success of NAM therapy depends on the experience of the clinicians, co-operation of the parents, and an infant that is healthy to undergo the treatment. In patients with cleft lip and palate who are medically compromised, NAM may not be a practical treatment option.

The objective of the present study was to use CAD/CAM technology to design a nasal molding device without an intra-oral palate component for infants with clefts and with underlying medical conditions. In this study, a new method is presented for pre-surgical nasal molding without the use of an intra-oral alveolar molding acrylic plate. The iNM device can also be used effectively in patients with complete clefts with varying alveolar gaps, bilateral clefts from mild to severe, and incomplete cleft of the lip and significant nasal deformity. The appliance can be made in mass-production and comes in different sizes or tailor-made for each patient, specifically it can be made asymmetrically to fit each nostril or build-in overcorrection. If no customization is needed, the standard size iNM device can be delivered at the initial visit. The iNM device is well tolerated by an infant and requires significantly less effort from the caregiver as the treatment protocol requires less in-office visits for adjustments. The iNM device does not interfere with feeding and allows proper force vectors, and is simple to use. Horizontal lip taping helps facilitate passive alveolar molding, which is ideal for centers that do not perform gingivoperioplasty. Since there is no intra-oral plate, ulceration in the oral cavity can be avoided with the iNM device.

In conclusion, the pre-surgical iNM therapy is simple to use and highly effective in molding the nasal cartilages and in improving nasal asymmetry prior to primary lip and nose repair. Without the use of an intra-oral acrylic plate, the horizontal lip plate indirectly molds the alveolar segments. The pre-surgical iNM is an ideal treatment alternative for nasal molding in patients with clefts and even in patients when NAM therapy cannot be used or when only nasal molding is indicated in patients with incomplete cleft.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A nasal molding device, comprising:
a pair of nasal molding stents comprising a first and a second molding stent, each molding stent having a substantially tubular shape comprising a superior end extending for a length to an inferior end;
a columella bridge laterally attached to the inferior end of each molding stent, the columella bridge having a width; and
a lip band attached to the columella bridge and the inferior end of each molding stent, the lip band having a substantially planar shape comprising a length extending between opposing lateral ends, a height extending from a superior to an inferior direction, and a thickness;
wherein each molding stent comprises a molding stent axis aligned in parallel with the length of each molding stent, and the lip band comprises a lip band axis aligned in parallel with the width of the lip band.

2. The device of claim 1, wherein each molding stent comprises a superior width and an inferior width.

3. The device of claim 2, wherein the superior width and the inferior width are each between about 1 and 15 mm.

4. The device of claim 2, wherein the inferior width of the first molding stent is equal to the inferior width of the second stent.

5. The device of claim 2, wherein the inferior width of the first molding stent is not equal to the inferior width of the second stent.

6. The device of claim 1, wherein the length of each molding stent is between about 5 and 20 mm.

7. The device of claim 1, wherein the superior end of each molding stent has a Shore hardness that is less than the inferior end of each molding stent.

8. The device of claim 6, wherein the Shore hardness of the superior end is between about 0 Shore OO and 100 Shore OO.

9. The device of claim 6, wherein the Shore hardness of the inferior end is between about 0 Shore OO and 100 Shore OO.

10. The device of claim 1, wherein the molding stent axis and the lip band axis form an angulation between about 90° and 180°, such that each molding stent is angled in a posterior direction.

11. The device of claim 10, wherein the angulation is between about 120° and 150°.

12. The device of claim 1, wherein the molding stent axis of the first molding stent is aligned in parallel with the molding stent axis of the second molding stent.

13. The device of claim 1, wherein the molding stent axis of the first molding stent is not aligned in parallel with the molding stent axis of the second molding stent.

14. The device of claim 1, wherein the width of the columella bridge is between about 1 and 15 mm.

15. The device of claim 1, wherein the length of the lip band is between about 20 and 100 mm.

16. The device of claim 1, wherein the height of the lip band is between about 1 and 15 mm.

17. The device of claim 1, wherein the thickness of the lip band is between about 0.5 and 5 mm.

18. The device of claim 1, wherein each lateral end comprises a connector configured to receive an elastic band, the connector being selected from the group consisting of: a loop connector, a button connector, and a hooked connector.

19. The device of claim 1, wherein the height of the lip band curves in a superior direction at a position directly inferior to an attachment between the lip band and the columella bridge.

20. The device of claim 1, wherein the length of the lip band comprises a curvature, such that each lateral end curves in a posterior direction.

21. The device of claim 1, further comprising one or more retainers, each retainer comprising a planar shape having a top surface comprising one or more connectors and a bottom surface configured to adhere to a subject's face or head.

22. The device of claim 21, wherein the one or more connectors are each configured to receive an elastic band, each connector being selected from the group consisting of: a loop connector, a button connector, and a hooked connector.

23. A method, comprising the steps of:
providing a first nasal molding device comprising a pair of nasal molding stents comprising a first and a second molding stent, each molding stent having a substantially tubular shape comprising a superior end extending for a length to an inferior end; a columella bridge laterally attached to the inferior end of each molding stent, the columella bridge having a width; and a lip band attached to the columella bridge and the inferior end of each molding stent, the lip band having a substantially planar shape comprising a length extending between opposing lateral ends, a height extending from a superior to an inferior direction, and a thickness; wherein each molding stent comprises a molding stent axis aligned in parallel with the length of each molding stent, and the lip band comprises a lip band axis aligned in parallel with the width of the lip band;
affixing the first device to a subject.

24. The method of claim 23, further comprising the steps of:
removing the first device from the subject;
providing a second nasal molding device comprising a pair of nasal molding stents comprising a first and a second molding stent, each molding stent having a substantially tubular shape comprising a superior end extending for a length to an inferior end; a columella bridge laterally attached to the inferior end of each molding stent, the columella bridge having a width; and a lip band attached to the columella bridge and the inferior end of each molding stent, the lip band having a substantially planar shape comprising a length extending between opposing lateral ends, a height extending from a superior to an inferior direction, and a thickness; wherein each molding stent comprises a molding stent axis aligned in parallel with the length of each molding stent, and the lip band comprises a lip band axis aligned in parallel with the width of the lip band;
affixing the second device to the subject; wherein
the columella bridge width of the second device is less than the columella bridge width of the first device;
the distance between the superior ends of the molding stents of the second device is less than the distance between the superior ends of the molding stents of the first device; or
the wall thicknesses of the pair of nasal molding stents of the second device is greater than the wall thicknesses of the pair of nasal molding stents of the first device.

25. The method of claim 23, further comprising the steps of:
providing one or more retainers, each retainer comprising a planar shape having a top surface comprising one or more connectors and a bottom surface configured to adhere to the subject's face or head;
adhering the one or more retainers to the skin of the subject; and
attaching one or more elastic bands from the one or more connectors of the retainers to the nasal molding device.
